(19) United States
(12) Patent Application Publication
(10) Pub. No.: US 2004/0166115 A1
(43) Pub. Date: Aug. 26, 2004

(54) Use of multi-specific, non-covalent complexes for targeted delivery of therapeutics

(75) Inventors: Gary L. Griffiths, Morristown, NJ (US); Serengulam V. Govindan, Summit, NJ (US); Hans J. Hansen, Picayune, MS (US)

Correspondence Address: HELLER EHRMAN WHITE & MCAULIFFE LLP, 1666 K STREET,NW, SUITE 300, WASHINGTON, DC, 20006, US

(73) Assignee: Immunomedics, Inc.

(21) Appl. No.: 10/714,391

(22) Filed: Nov. 17, 2003

Related U.S. Application Data
(60) Provisional application No. 60/426,379, filed on Nov. 15, 2002.

(51) Int. Cl. A61K 39/395

(52) U.S. Cl. 424/178.1

(57) ABSTRACT

The invention relates to a method for treating target cells, tissues or pathogens in a subject, comprising administering a non-covalently bound complex which comprises a multispecific targeting protein and a hapten-enzyme covalent conjugate. The invention further relates to kits comprising the non-covalently bound complex or the components to prepare the complex.

### CROSS-REFERENCE TO RELATED APPLICATIONS

This application claims priority under 35 U.S.C. 119(e) to U.S. Provisional Application Serial No. 60/426,379, filed Nov. 15, 2002, the disclosure of which is incorporated by reference herein in its entirety.

### FIELD OF THE INVENTION

The invention relates to a method, a composition and a kit for delivering therapeutic agents to subjects.

### BACKGROUND OF THE INVENTION

The selective delivery of therapeutic agents to diseased tissue, in vivo, remains a major challenge in the interests of improved therapeutic outcomes. It must be appreciated that although much of the following discussion of the invention is specified toward anti-cancer treatments, any disease state amenable to treatment with drugs or prodrugs could be addressed in the same way. In the case of cancers, standard chemotherapies have depended, in general, upon the enhanced uptake of toxic drugs by fast-growing diseased cells, in relation to most normal cells. However, this has been found to be only of limited value, and normal cell toxicities are often reached before a decisive therapeutic effect against the cancer can be obtained. Typically, those normal cells that divide the fastest are most prone to the adverse effects of chemotherapy agents.

Several different approaches are being taken that seek to improve the therapeutic outcomes resulting from anti-cancer drug therapies. One is the use of mixtures or ‘cocktails’ of drugs, with component drugs often chosen for their effects on different aspects of cell metabolism. A second is the encapsulation of drugs into carriers such as liposomes or the attachment of drugs to long-circulating polymers. This approach extends drug half-life in serum, and generally allows for a greater proportion of the administered drug to be deposited at the target site. A third approach can be viewed as an advance on the second approach, in that drugs are attached to specific targeting agents such as monoclonal antibodies or peptides. These agents are able to specifically accrete at a target due to their binding to an antigen or receptor, respectively, which has been upregulated or specifically produced by the target cells.

A disadvantage with the aforementioned approaches is the tendency of drugs to lose potency upon conjugation to a polymer, peptide or monoclonal antibody (MAb). Numerous articles have described methods of drug conjugation that seek to preserve drug activity while forming a stable bio-conjugate. Unfortunately many drug-carrier conjugates also dissociate when subjected to the challenge of an in vivo serum environment. Moreover, tumor uptakes of the drug are reduced while non-specific toxicity to normal tissues are often increased.

An advanced method for delivering a drug to a disease site in a less toxic and more efficient and efficacious manner, is the use of antibody-directed enzyme prodrug therapy (ADEPT). See, for example, U.S. Pat. No. 5,632,990 (Bagshawe). Originally, ADEPT depended on the use of a conjugate of an antibody and an enzyme to localize the latter to a site of disease. Such an approach had several drawbacks, including loss of antibody and/or enzyme activity upon conjugation, and high residual levels of circulating MAb-enzyme conjugate in the bloodstream due to the long-circulating MAb. The latter, in turn, resulted in excessive enzyme activity in circulation upon administration of the prodrug, which was cleaved by enzyme in the bloodstream, generating high levels of active drug, and high levels of non-specific drug toxicity.

Later, the use of bispecific antibodies (bsAbs) was suggested for application to the ADEPT method. In this approach, a bispecific antibody targeting both a disease-associated antigen with one arm, and an epitope on an enzyme with a second arm would be given to a subject, followed some time later by the enzyme in question, and finally by the prodrug that the enzyme was active against. This invention comprises a three-step delivery system, absent any clearing agents. Difficulties encountered in the practice of this ADEPT method in the second capture step, that is by the second arm of the bsAb against the enzyme epitope, perhaps due to low affinity of this antibody-antigen complex, led to protocols where the bsAb and the enzyme were mixed together, and administered as a single complex, followed later by the prodrug. This altered approach comprises a two-step delivery system, absent any clearing agents. However, this modified ADEPT method remained fraught with problems preventing its ultimate wide application in patients. These included the utility of the targeting arm of the bsAb, bsAb preparation issues, binding affinity of the second (anti-enzyme epitope) arm of the bsAb, choice of prodrug, efficiency of prodrug cleavage by the enzyme, and, not least, presence of active enzyme in non-target tissues at the time of prodrug administration. The latter leads to unwanted cleavage of prodrug in normal tissues, and, subsequently, untoward toxicity due to the generation of active drug in those tissues. A particular problem was encountered in the cleavage of drug to prodrug in the circulation, by active enzyme.

A continuing need therefore exists for methods and compositions that are able to selectively deliver therapy agents to a disease site using an ADEPT approach, without undue dissociation of bsAb and enzyme, and without adversely affecting a therapeutic agent's potency.

### SUMMARY OF THE INVENTION

The inventors have surprisingly discovered that when a multispecific targeting protein (e.g., a bi-specific monoclonal antibody) is pre-mixed with a hapten-enzyme covalent conjugate, the resulting complex can be used to localize the enzyme specifically to the site of disease via the targeting arm of the multispecific antibody. The strength of complex binding between the secondary [hapten-binding] arm of the multispecific antibody and the hapten-enzyme conjugate is sufficient to hold the enzyme in a position and concentration suitable for successful ADEPT. The non-covalently bound complex of bsAb/hapten-enzyme remains in circulation for an extended period, showing the stability of the binding between the hapten-binding arm of the bsAb and the hapten-enzyme conjugate. Because the secondary arm of the bsAb is raised against a carefully selected hapten, rather than a non-defined epitope on a particular enzyme, the secondary arm of the bsAb can be carefully screened to have the optimum binding properties. In addition, the same secondary arm-containing bsAb may be used with different enzymes, since the same recognition hapten is being recognized, once the hapten is substituted onto a different enzyme. Such a non-covalently bound complex represents an example of a superior general method for delivery of therapy agents, using ADEPT, to disease tissue targets. This new ADEPT methodology can be adopted to circumvent the aforementioned problems with covalent drug-carrier conjugates, as well as problems seen with earlier versions of the ADEPT concept.

In one aspect, the invention relates to a method for treating target cells, tissues or pathogens in a subject, such as a mammal, comprising administering in sequence:

a) a therapeutically effective amount of a non-covalently bound complex to said subject thereby forming a target-tissue-localized complex;

wherein said non-covalently bound complex comprises a multispecific targeting protein comprising at least one target-binding site and one hapten-binding site, and a hapten-enzyme covalent conjugate;

wherein said at least one target-binding site is capable of binding to at least one complementary binding moiety on the target cells, tissues or pathogens or on a molecule produced by or associated with said target cells, tissues or pathogens; and

wherein said hapten-binding site is non-covalently bound to the hapten-enzyme covalent conjugate;

b) optionally, a clearing agent; and

c) a chemotherapeutic drug or prodrug, capable of being converted to an active drug by the target-tissue-localized complex. More specifically, the chemotherapeutic drug is converted to an active drug by a target-tissue-localized complex that is an enzyme.

In another aspect, the invention relates to a kit comprising, in suitable containers:

a) a multispecific targeting protein, comprising at least one target-binding site and a hapten-binding site, pre-mixed with a hapten-enzyme conjugate; and

b) a chemotherapeutic prodrug.

In yet another aspect, the invention relates to a kit comprising, in separate, suitable containers:

a) a multispecific targeting protein, comprising at least one target-binding site and a hapten-binding site;

b) a hapten-enzyme conjugate; and

c) a chemotherapeutic prodrug;

wherein said multispecific targeting protein, comprising at least one target-binding site and a hapten-binding site and said hapten-enzyme conjugate are mixed immediately prior to use.

In yet another aspect, the invention relates to a method of making a stable non-covalently bound complex that is capable of localizing to a target cell, tissue, or pathogen comprising admixing a multispecific targeting protein comprising at least one target-binding site and a hapten-binding site, and a hapten-enzyme covalent conjugate;

wherein said at least one target-binding site is capable of binding to at least one complementary binding moiety on said target cells, tissues or pathogens or on a molecule produced by or associated with said target cells, tissues or pathogens; and

wherein said hapten-binding site is capable of stably and non-covalently binding said hapten-enzyme conjugate; thereby making a stable non-covalently bound complex.

In still another aspect, the invention relates to a method of treating a subject, comprising administering a therapeutically effective amount of a non-covalently bound complex, said non-covalently bound complex resulting from the pre-mixing of said multi-specific targeting protein and a hapten-enzyme conjugate, prior to administration to said subject.

### DETAILED DESCRIPTION

As used herein, the term “subject” refers to any mammal. In one embodiment, the mammal is a human.

I. Non-covalently Bound Complex: A Multispecific Targeting Protein and a Hapten-enzyme Conjugate.

As used herein, the term “targeting protein” is a multispecific binding protein, such as a bispecific antibody, or a recombinantly produced antigen-binding molecule in which two or more of the same or different natural antibody, single-chain antibody or antibody fragment segments with different specificities are linked. The valency of the targeting protein refers to the total number of binding arms or sites the targeting protein has to a particular antigen or epitope. Thus, depending on the total number of binding arms or sites the targeting protein has to an antigen or epitope, the targeting protein may be monovalent, bivalent, trivalent or multivalent. A multivalent targeting protein has the advantage of multiple interactions in binding to an antigen, thus increasing the avidity of binding to said antigen.

The specificity of the targeting protein refers to how many antigens or epitopes a targeting protein is able to bind. Thus, the targeting protein may be monospecific, bispecific, trispecific or multispecific. A multispecific targeting protein has the advantage of multiple interactions in binding to separate antigens, thus increasing the avidity of binding to the cellular target. Using these definitions, a natural antibody (e.g., an IgG) is bivalent because it has two binding arms but is monospecific because it binds to one antigen. Monospecific (to target cell), multivalent targeting proteins have more than one binding site for an epitope, but only bind with the same epitope on the same antigen. A second example of a monospecific, multivalent targeting protein is a diabody with two binding sites reactive to the same antigen. The targeting protein may comprise both multivalent and multispecific combinations of different antibody components including multiple copies of the same antibody components.

Examples of multivalent target binding proteins are described in Patent Appl. Serial No. 60/220,782. Multivalent target binding proteins have been made by cross-linking several Fab-like fragments via chemical linkers. See U.S. Pat. Nos. 5,262,524; 5,091,542 and Landsdorp et al. Euro. J. Immunol. 16: 679-83 (1986). Multivalent target binding proteins also have been made by covalently linking several single chain Fv molecules (scFv) to form a single polypeptide. See U.S. Pat. No. 5,892,020. A multivalent target binding protein which is basically an aggregate of scFv molecules has been disclosed in U.S. Pat. Nos. 6,025,165 and 5,837,242. A trivalent target binding protein comprising three scFv molecules has been described in Krott et al. Protein Engineering 10(4): 423-433 (1997).

In a preferred aspect of the invention, the multivalent and multipsecific targeting protein is a bispecific antibody. Such a targeting protein is exemplified by a Fab′×Fab′ fragment, wherein the first Fab′ fragment binds to an anti-tumor cell epitope, and the second Fab′ fragment binds to a low molecular weight hapten. In this embodiment the two distinct specificity Fab′ fragments can be linked through their hinge region thiol groups using commercially available cross-linkers and methods well-known in the art. A second targeting protein is exemplified by a F(ab′)₂×Fab′ fragment, wherein the divalent F(ab′)₂ fragment binds to an anti-tumor cell epitope, and the single-valent Fab′ fragment binds to a low molecular weight hapten. Similarly, a third targeting protein is exemplified by an intact IgG×Fab′ fragment, wherein the divalent IgG binds to an anti-tumor cell epitope, and the single-valent Fab′ fragment binds to a low molecular weight hapten. Other combinations of specificity and valency of both the anti-target cell arm and the anti-hapten arm may be readily envisaged.

In one preferred aspect of the invention, the multivalent and multispecific (to cellular target and to hapten) targeting protein is a bivalent anti-antigen and monovalent anti-hapten bispecific antibody. Bivalency toward the cellular target better retains the ability of the composition to remain on the cell surface, or associated with the cell for an extended period of time. Monovalency to the hapten limits the amount of cross-linking that can take place with a hapten-enzyme conjugate, and therefore regulates final molecular size. A specific example of such an agent is an anti-CEA×anti-indium-DTPA F(ab′)₂×Fab′ bispecific antibody, wherein CEA refers to carcinoembryonic antigen and DTPA refers to diethylenetriaminepentaacetic acid. Further examples will be discussed below.

The target-binding site of a disease-targeting antibody arm is capable of binding to a complementary binding moiety on the target cells, tissues, pathogens or on a molecule produced by, or associated with, the target cell tissue or pathogen. In a preferred aspect of the present invention, the pathogen is selected from the group consisting of a virus, a fungus, a parasite and a bacterium. The complementary binding moieties that are contemplated in one aspect of the present invention include, but are not limited to tumor-associated antigens (TAAs), wherein said antigens are selected from the group consisting of AFP (alpha fetal protein), HCG (human chorionic gonadotropin), EGP-1, EGP-2, CD37, CD74, colon-specific antigen-p (CSAp), carcinoembryonic antigen (CEA), CD19, CD20, CD21, CD22, CD23, CD30, CD74, CD80, HLA-DR, Ia, MUC 1, MUC 2, MUC 3, MUC 4, EGFR, HER 2/neu, PAM-4, TAG-72, EGP-1, EGP-2, A3, KS-1, Le(y), S100, PSMA, PSA, tenascin, folate receptor, VEGFR, necrosis antigens, IL-2, T101 and MAGE. Specific targeting antibodies include, but are not limited to: MN-14 (anti-carcinoembryonic antigen), Mu-9 (anti-colon specific antigen-P), LL2 (anti-CD22), LL1 (anti-CD74), hA20 (anti-CD20) RS7 (anti-epithelial glycoprotein). Such antibodies encompass chimeric, humanized and human antibodies containing the same CDRs as their corresponding murine antibodies. See U.S. Pat. Nos. 5,874,540; 5,789,554 and 6,187,287. See also pending U.S. patent application Ser. Nos. 10/116,116; 09/337,756; No. 60/360,259; and No. 60/356,132.

The multispecific targeting protein also has an arm referred to as the hapten-binding site or arm. The hapten-binding site is typically an antibody or a hapten binding antibody fragment and is raised against a defined a low molecular weight hapten. Such low molecular weight haptens include agents such as DTPA (diethylenetriaminepentaacetic acid), DOTA (1,4,7,10-tetraazacyclododecane-N,N′,N″,N′″-tetraacetic acid) and HSG (histamine succinyl glycine moiety):

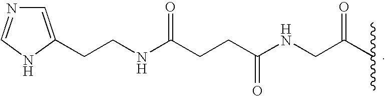

The antibodies are generally raised after binding of the low molecular weight hapten to an immunogen (e.g., keyhole limpet hemocyanin, or another foreign protein) using methods well know in the art. Specific examples of antibodies that can comprise the hapten-binding site of a multispecific targeting protein include MAbs 734 (anti-diethylenetriaminepentaacetic acid-indium complex; anti-DTPA), 679 (anti-histaminyl succinyl glycyl; anti-HSG) and LG1 (anti-DOTA).

Aside from the MAbs disclosed herein, it can be appreciated that other MAbs can be raised to any hapten or drug by standard methods of making mAbs known to a person skilled in the art. For instance, it is possible to attach, a hapten such as HSG to an immunogenic stimulator or adjuvant such a keyhole limpet hemocyanin, and inject the conjugate into immunocompetent animals. Multiple injections are often employed. It must be appreciated that such an approach can lead to several different antibodies with slightly different specificities against the hapten in question, such as HSG. MAbs can recognize different sub-parts of the HSG structure, or different conformations. MAbs may also be obtained that recognize a little more than just the HSG molecule itself, such as recognizing an HSG moiety only when attached to an epsilon amino group of lysine, if indeed, the HSG was initially linked to the KLH (for example) by attachment to an epsilon lysyl amino group on the latter immunogenic protein. Without wishing to be exhaustive, these general procedures and results are well known in the art. It is also then well known art for the isolation of spleen cells producing antibodies from these immunized animals, and their subsequent fusion with myeloma cell lines, to generate hybridomas secreting anti- hapten antibodies. See Kohler G. and Milstein C., Eur. J. Immunol. 6:511-9 (1976); Kohler G. et al., Eur. J. Immunol. 6:292-5 (1976); and Kohler G. and Milstein C. Nature 256:495-7 (1975).

Multispecific targeting proteins can be prepared chemically from antibodies that have differing specificity by well-known reactions. Typically, one MAb is activated by reaction with a cross-linking agent, with the latter chosen to react at the first MAb's lysine, reduced cysteine, or oxidized carbohydrate residues. After purification, the activated first MAb is mixed with the second MAb, which then reacts specifically with a second functionality of the original cross-linking agent; most notably via the second MAb's lysine, reduced cysteine or oxidized carbohydrate residues. Multispecific targeting proteins can also be prepared, somatically by the quadroma technique. The quadroma technique is a technique wherein a cell line expressing both arms of the bispecific antibody is produced and grown in culture to secrete the bsMAb. Finally, bsMAbs can also be produced conveniently by modern techniques of molecular biology. See, for example Colman, A., Biochem. Soc. Symp. 63: 141-147 (1998); U.S. Pat. No. 5,827,690; and Published U.S. Application 20020006379.

BsAbs of the types exemplified above can be pre-mixed with several different hapten-enzyme conjugates to produce and deliver an effective therapy agent, after appropriate prodrug administration, depending on what the pertinent arm of the bsAb has been raised against. In a preferred embodiment, the enzyme contained in the hapten-enzyme covalent conjugate is selected from the group consisting of an esterase, carboxylesterase, carboxypeptidase, amidase, glucoronidase and galactosidase. Most preferably, the esterase is a carboxylesterase selected from the group consisting of rat, mouse, rabbit, porcine and human carboxylesterase. The enzyme may be produced by recombinant techniques well know in the art (Wolfe, et al. 1999). The enzyme may be produced in yeast, bacteria, plants, insect or animal cells. Preferably, the enzyme has been modified to enhance its catalytic properties (Wolfe et al, 1999). The modification may be performed via site-directed mutagenesis. See U.S. Pat. Nos. 5,352,594 and 5,912,161 for a general discussion of site-directed mutagenesis. In any case, the desired effect of the mutagenesis is to reduce the Michaelis constant of the enzyme, enabling more efficient enzyme activity at lower concentrations of prodrug substrate. It is preferred that the multispecific targeting protein binds to both its antigenic target and to its hapten target via the target binding site and the hapten binding site, respectively, with a dissociation constant of at least 10⁻⁷; more preferably at least 10⁻⁹.

Haptens can be attached to enzymes in several ways. For instance, the DTPA hapten can be coupled to the enzyme carboxylesterase at certain individual positions on the enzyme to give the hapten-enzyme covalent conjugate. Most simply the commercially available precursor DTPA dianhydride is added to a solution of enzyme in an appropriate buffer, at pH 7-9. After a reaction of from 1-16 hours, using an appropriate molar excess of DTPA-dianhydride, one or more units of DTPA are attached to the enzyme, by reaction of the latter's lysyl residues with one anhydride group of the precursor. The DTPA-enzyme conjugate is separated from unreacted, hydrolyzed DTPA and buffer components by standard methods for effecting such separations, such as ammonium sulfate precipitation, diafiltration or size-exclusion or ion-exchange chromatography. To obtain the bsAb-hapten-enzyme conjugate the hapten-enzyme covalent conjugate is then mixed with a bsAb, such as MN-14×734 bsAb (anti-CEA×anti-DTPA) to give a non-covalently bound complex wherein the target-binding site capable of binding to a complementary binding moiety on the target cells is MN-14. A typical complex might then be: MN-14×734 bsAb/DTPA-carboxylesterase. The bsAb and the hapten-enzyme conjugate may be mixed together in ratios of from 5:1 to 1:5, or more preferably in ratios of from 2:1 to 1:2. The complex may be made immediately prior to use, or it may be made in advance and stored under appropriate conditions until required. It may also be frozen for shipping and future use, or formulated for long-term storage by lyophilization. Such methods are well known in the art.

The hapten-enzyme conjugate may also be made using an alternate approach, designed to attach two hapten recognition units to the enzyme in one chemical reaction. In this approach, an intermediate comprising two such hapten recognition units is attached to a short peptide carrier backbone that also incorporates a group for activation and coupling to the enzyme. The agent has the general formula: -X-peptide(-X)-(reactive group); where the peptide is 2-10 amino acid residues in length, preferably 2-5 amino acid residues in length, most preferably, the peptide is 3-4 amino acid residues in length; the X moieties are recognition hapten residues mentioned previously, exemplified by In-DTPA, DOTA or HSG sub-units; and the reactive moiety comprises a functionality that can be coupled to the enzyme without interference from the rest of the bivalent recognition conjugate. An Example of such a structure is Ac-NH-Lys(HSG)-Tyr-Lys(HSG)-COOH; a tripeptide of two lysyl residues and one tyrosyl residue, linked together by amide bonds, and blocked on its alpha amino group by an unreactive group such as an acetyl residue. The amino acids may be in the L- or the D-conformation. Each lysyl residue, though its epsilon amino group, is attached to a HSG recognition unit. The reactive moiety in this instance is a carboxyl group that can be further activated via an anhydride, active ester or other such activating agent, for coupling to free amino groups on an enzyme.

An second similar example of such a structure is 4(4-N-maleimidomethyl)cyclohexanecarboxyamido-Lys(DTPA)-Tyr-Lys(DTPA)-CONH₂; a tripeptide of two lysyl residues and one tyrosyl residue, linked together by amide bonds, and blocked on its carboxyl terminal group by an unreactive group such as an amide residue. The amino acids may be in the L- or the D-conformation. Each lysyl residue is attached to a DTPA recognition unit. The reactive moiety in this instance is a maleimido group that might be coupled to free thiol groups on an enzyme, wherein the free thiol groups are present endogenously, or are placed there by prior reaction of the enzyme with a thiolating agent such as Traut's reagent.

Many more such compositions can be envisaged as useful within the context of the current invention. See for example published U.S. application 20020006379 and pending U.S. application Ser. No. 09/337,756.

After administration, localization to the site of disease, and substantial. clearance from normal tissues of the bsAb/hapten-enzyme complex, a drug or prodrug substrate to the enzyme in question may be given. For example, with a CEA-expressing tumor, the above MN-14×734 bsAb, pre-complexed with DTPA-carboxylesterase is given, allowed to localize to CEA-expressing tumor sites, and clear normal tissues, before the prodrug CPT-11 (irinotecan) (a substrate for carboxylesterase) is given. The non-covalently bound bsAb-hapten-enzyme complex that has localized at the tumor, activates the subsequently administered prodrug specifically at the site of the tumor. A variety of chemotherapeutic agents or prodrugs of chemotherapeutic agents may be used in the practice of the preferred embodiments of the present invention for treatment of subjects. Such chemotherapeutic agents include, but are not limited to, adriamycin, actinomycin, calicheamycin, epothilones, maytansine, mitomycin, carminomycin, daunomycin, doxorubicin, tamoxifen, taxol and other taxanes, taxotere, vincristine, vinblastine, vinorelbine, etoposide (VP-16), 5-fluorouracil (5FU), cytosine arabinoside, cyclophohphamide, thiotepa, methotrexate, camptothecin, actinomycin-D, mitomycin C, cisplatin (CDDP), aminopterin, combretastatin(s), neomycin, and podophyllotoxin(s). Anti-metabolites such as cytosine arabinoside, amethopterin; anthracyclines; vinca alkaloids and other alkaloids; antibiotics, demecolcine; etopside; mithramycin; and other anti-tumor alkylating agents are also contemplated for use in the present invention.

Preferred prodrugs of the preferred embodiments are those derived from the drugs selected from the group consisting of camptothecin, doxorubicin, taxol, actinomycin, maytansine, calicheamycin and epothilones.

The term “prodrug” refers to an agent that is converted into the parent drug in vivo. Prodrugs are often useful because, in some situations, they may be easier to administer than the parent drug. The prodrug, for instance, may be bioavailable by oral administration whereas the parent drug is not. The prodrug may also have improved solubility in pharmaceutical compositions over the parent drug. A prodrug may be converted into the parent drug by various mechanisms, including enzymatic processes and metabolic hydrolysis. See Harper, “Drug Latentiation” in Jucker, ed. Progress in Drug Research 4:221-294 (1962); Morozowich et al., “Application of Physical Organic Principles to Prodrug Design” in E. B. Roche ed. Design of Biopharmaceutical Properties through Prodrugs and Analogs, APHA Acad. Pharm. Sci. (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of β-Lactam antibiotics,” Pharm. Biotech. 11,:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenytoin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Adv. Drug Delivery Rev. 8(1): 1-38 (1992); Fleisher et al. “Improved oral drug delivery: solubility limitations overcome by the use of prodrugs”, Adv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, J. Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” J. Chem. Soc., Chem. Commun., 875-877 (1991); Friis and Bundgaard, “Prodrugs of phosphates and phosphonates: Novel lipophilic alpha-acyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989).

A clearing agent may be optionally added after administration of the non-covalently bound multispecific antibody-hapten-enzyme complex to a subject. The clearing agent is preferably an antibody directed against an epitope of the multispecific targeting protein/hapten-enzyme complex. Most preferably, the clearing agent is an anti-idiotypic antibody, a carbohydrate-derivatized anti-idiotypic antibody or a galactosylated anti-idiotypic antibody to the multispecific targeting protein.

Non-proteinaceous polymers can also serve as the backbone onto which other drugs or prodrugs useful in the present invention may be attached. The polymeric material serves to detoxify and solubilize the active drug. For instance a co-polymer consisting of (Lys)ₘ-(Glu)ₓ-(Taxol)_{y} (wherein m is an integer from 10-500, n is an integer from 10-500, and y is an integer from 1-50) can be applied in this manner, being given after the injection, localization and clearance of the multipsecific antibody-hapten-enzyme complex. In this instance, the enzyme in question would comprise an esterase, capable of cleaving the ester bond between taxol and the gamma-carboxyl groups of the multi-glutamic acid units. This type of prodrug is based on the utility of polymeric material to carry active drugs in circulation for an extended period of time. See Auzenne et al., Clin Cancer Res. 8: 573 (2002) and Li et al., Cancer Res., 1998. Other drugs, such as camptothecins may be used in a similar manner, and other polymers such as poly-N-(2-hydroxypropyl)methacrylamide (HPMA) may also be applied as carriers. The invention also contemplates the incorporation of unnatural amino acids, e.g., D-amino acids, into the non-proteinaceous polymers. The invention further contemplates other backbone structures such as those constructed from non-natural amino acids. See for example, published U.S. application 20030026764.

In another aspect, the invention relates to a method of making a stable target-tissue-localized complex comprising pre-mixing a multispecific targeting protein comprising at least one target-binding site and a hapten-binding site, and a hapten-enzyme covalent conjugate;

wherein said at least one target-binding site is capable of binding to at least one complementary binding moiety on the target cells, tissues or pathogens or on a molecule produced by or associated with said target cells, tissues or pathogens; and

wherein said hapten-binding site is capable of stably and non-covalently binding a hapten-enzyme conjugate; thereby forming a stable target-tissue-localized complex.

II. Formulations and Kits

The multispecific targeting protein and hapten-enzyme covalent conjugate that comprises the non-covalently bound complex preferably also comprise a pharmaceutically acceptable carrier or excipient. A pharmaceutically acceptable carrier is a carrier or diluent that does not cause significant irritation to an organism and does not abrogate the biological activity and properties of the administered compound. An excipient is an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycol derivatives.

One aspect of the present invention relates to a kit comprising, in suitable containers, separate or together:

a) a multispecific targeting protein, comprising a target tissue-binding site and an hapten-binding site, pre-mixed with a hapten-enzyme conjugate; and

b) a chemotherapeutic prodrug.

Another aspect of the invention relates to a kit comprising, in separate, suitable containers:

a) a multispecific targeting protein, comprising at least one target-binding site and a hapten-binding site;

b) a hapten-enzynie conjugate; and

c) a chemotherapeutic prodrug;

wherein said multispecific targeting protein, comprising at least one target-binding site and a hapten-binding site and said hapten-enzyme conjugate are mixed immediately prior to use.

The kit, may comprise the non-covalently bound complex and a pharmaceutically acceptable carrier or excipient. Likewise, the kit may comprise the prodrug in a pharmaceutically acceptable carrier or excipient. In a preferred embodiment of the present invention, the kit comprises a bispecific antibody such as anti-CEA×anti-indium-DTPA-F(ab′)₂. The bispecific antibody is mixed with an equimolar amount of the enzyme-hapten conjugate DTPA-carboxylesterase. The kit can contain from about 1-10,000 mg of the mixture. The kit can be stored as a sterile solution, frozen at −20 to −80° C., or it can be lyophilized to powder form for long-term storage. In one embodiment, these formulations could include a preformed single vial kit comprising multispecific antibody-hapten-enzyme conjugate, or two separate vials containing multispecific antibody, and hapten-enzyme, respectively, which are then mixed prior to administration. From a formulation and stability perspective, the hapten-enzyme may be kept separate for long-term storage, and these determinations need to be made empirically, for each individual application of the technology.

III. Dosage

An amount of the non-covalently bound complex necessary for treating target cells, tissues or pathogens in a subject when provided to a subject is a “therapeutically effective” amount. In order to treat the target cells, tissues or pathogens, it is desirable to provide from about 0.001 to about 10,000 μmol of non-covalently bound complex per kilogram of subject weight. This dosage may be administered over a period from about 1 minute to about 4 hours, by any suitable means, but prior to the administration of the chemotherapeutic drug or prodrug. The non-covalently bound complex of the present invention may be dissolved in any physiologically tolerated liquid in order to prepare an administrable amount. It is preferable to prepare such a solution of the non-covalently bound complex by dissolving the non-covalently bound complex in normal saline, phosphate buffered saline (pH from about 5 to about 8), acetate buffered saline (pH from about 4 to about 7), phosphate buffer (pH from about 5 to about 8), or acetate buffer (pH from about 4 to about 7). Buffered concentrations in the 0.02 to 2 molar range are acceptable.

An amount of the chemotherapeutic drug or prodrug necessary to treat target cells, tissues or pathogens in a subject when provided after the administration of the non-covalently bound complex to a subject is a “therapeutically effective” amount. In order to treat the target cells, tissues or pathogens, it is desirable to provide from about 0.001 to about 10,000 μmol of non-covalently bound complex per kilogram of subject weight. This dosage may be administered over a period from about 1 minute to about 4 hours, by any suitable means, but following the administration of the non-covalently bound complex. The chemotherapeutic drug or prodrug of the preferred aspects of the present invention may be dissolved in any physiologically tolerated liquid in order to prepare an administrable amount. It is preferable to prepare such a solution of the non-covalently bound complex by dissolving the non-covalently bound complex in normal saline, phosphate buffered saline (pH from about 5 to about 8), acetate buffered saline (pH from about 4 to about 7), phosphate buffer (pH from about 5 to about 8), or acetate buffer (pH from about 4 to about 7). Buffered concentrations in the 0.02 to 2 molar range are acceptable. Drugs or prodrugs may be administered in the ways that they are usually administered when given as independent active entities. For instance, hydrophobic drugs or prodrugs may be given in dextrose solutions or as admixtures with cremophor.

Suitable routes of administration of the non-covalently bound complex and the chemotherapeutic drug or prodrug include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. The preferred routes of administration are parenteral. Alternatively, one may administer the bsAb/enzyme-hapten complex and the drug or prodrug in a local rather than systemic manner, for example, via injection of the compound directly into a solid tumor.

The ordinary skilled artisan will appreciate that the pre-mixing of the multi-specific targeting protein and the hapten-enzyme conjugate, prior to administration to a subject, can be done with immediately before administration, or, it can be done well in advance.

IV. Treatment

In another aspect, the invention relates to a method of treating a subject, comprising administering a therapeutically effective amount of a non-covalently bound complex, said non-covalently bound complex resulting from the pre-mixing of said multi-specific targeting protein and a hapten-enzyme conjugate, prior to administration to a subject. Diseases that may be treated using the pre-mixed multi-specific targeting proteins and hapten-enzyme conjugates of the preferred embodiments of the present invention include, but are not limited to malignancies. These may include all solid and non-solid tumor cancers. In the case of the latter, B-cell cancers, or T-cell cancers can be treated (e.g., non-Hodgkins lymphoma, T-cell lymphoma or chronic lymphocytic leukemia). Equally, solid tumors may be treated using the current compositions and methods. These include, but are not limited to, adenocarcinomas and sarcomas. Major cancers of endodermally-derived digestive system epithelia, and cancers of the breast, prostate and lung are contemplated and treatable using this approach. In preferred applications diseases expressing antigens such as AFP (alpha fetal protein), HCG (human chorionic gonadotropin), EGP-1, EGP-2, CD37, CD74, colon-specific antigen-p (CSAp), carcinoembryonic antigen (CEA), CD19, CD20, CD21, CD22, CD23, CD30, CD74, CD80, HLA-DR, Ia, MUC 1, MUC 2, MUC 3, MUC 4, EGFR, HER 2/neu, PAM-4, TAG-72, EGP-1, EGP-2, A3, KS-1, Le(y), S100, PSMA, PSA, tenascin, folate receptor, VEGFR, necrosis antigens, IL-2, T101 and MAGE can be targeted with an appropriate antigen-targeting antibody arm on the multispecific antibody. Specific targeting antibodies include, but are not limited to: MN-14 (anti-carcinoembryonic antigen), Mu-9 (anti-colon specific antigen-P), LL2 (anti-CD22), LL1 (anti-CD74), hA20 (anti-CD20) RS7 (anti-epithelial glycoprotein-1). Such antibodies encompass chimeric, humanized and human antibodies containing the same CDRs as their corresponding murine antibodies.

Other diseases than cancer can also be targeted using these multispecific antibody/hapten-enzyme conjugates. For example, anti-CD19, 20, 22 and 74 antibodies can be used to treat immune dysregulation diseases and related autoimmune diseases, including Class III autoimmune diseases such as immune-mediated thrombocytopenias, such as acute idiopathic thrombocytopenic purpura and chronic idiopathic thrombocytopenic purpura, dermatomyositis, Sjögren's syndrome, multiple sclerosis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, sarcoidosis, ulcerative colitis, erythema multiforme, IgA nephropathy, polyarteritis nodosa, ankylosing spondylitis, Goodpasture's syndrome, thromboangitis ubiterans, (repeat), primary biliary cirrhosis, Hashimoto's thyroiditis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis and fibrosing alveolitis.

The following examples are meant to be illustrative of the methods, compositions and uses of the invention, and are not intended to be limitative.

### EXAMPLE 1

### Preparation of a Carboxylesterase-DTPA Conjugate

Two vials of rabbit liver carboxylesterase (about 8.5 mg protein content/vial) are reconstituted with 2.3 mL of 50 mM potassium phosphate buffer pH 7.5, and the solution is made 4.2 mM in DTPA using 0.1 mL of a 0.1 M stock solution of DTPA pH 6.7. The pH of the resultant solution is adjusted to be in the 7.7-7.8 range, and then reacted with 10 mg of cyclic DTPA dianhydride. After 1 h of stirring at the room temperature, the reaction mixture is passed through two successive SEC columns equilibrated in 0.1 M sodium phosphate pH 7.3. The eluate is further purified by preparative HPLC on a TSK G3000SW column using 0.2 M sodium phosphate pH 6.8, at 4 mL/min flow, as the eluent. The purified conjugate is made 0.1 M in sodium phosphate pH 6.8, and concentrated. The DTPA-to-carboxylesterase molar substitution ratio, determined by a metal-binding assay, is estimated to be in the range of 2.95-to-1 to 4.43-to-1.

### EXAMPLE 1A

### Preparation of hMN-14 IgG×734 Fab′ bispecific Antibody

HMN-14 IgG (8.45 mg, MW 150K) was derivatized with a 1.8-fold molar excess of sulfo-SMCC, at pH 7.21, for 45 minutes at ambient temperature. The product was purified by centrifuged SE column (‘spin column’) of Sephadex G50/80 in 0.1 M sodium phosphate, pH 6.5. The maleimide content was determined to be 0.93 moles per mole of IgG, by reacting with a known excess of 2-mercaptoethanol, followed by the determination of unused thiol by Ellman's assay. Separately, 734 F(ab′)₂ was reduced with 0.1 M cysteine (˜100-fold molar excess of cysteine) in 20 mM Hepes buffer-150 mM sodium chloride-10 mM EDTA, pH 7.3. The reduction was carried out for 50 min at 37° C. (bath) under argon flush. The reduced material was purified by two successive spin columns on Sephadex G50/80 in 0.1 M sodium phosphate-S mM EDTA, pH 6.5. The hMN-14-maleimide was reacted with 2-fold excess of 734 Fab′, and incubated at ambient temperature for 1 h. The conjugate was then reacted with a 40-fold molar excess of N-ethylmaleimide for 40 min. The material was subjected to preliminary purification on ‘spin column’ of Sephadex G50/80 in 0.1 M sodium phosphate, pH 7.3. The eluate from this purification was applied to a column of 3 mL of DTPA-Affigel, which was sequentially eluted with 0.2 M sodium phosphate pH 6.8 and 0.1 M EDTA, pH 3.8. Pooled EDTA fractions, dialyzed against 0.2 M sodium phosphate pH 6.8, with 2 buffer changes. The sample was applied to a preparative SE HPLC column (TSK G3000SW), with 0.2 M sodium phosphate pH 6.8 at 4 mL/min as running buffer. The major product was separated, and was found pure by SE HPLC (ret. time 9.58 min on analytical SE HPLC, 0.2 M sodium phosphate at 1 mL/min flow). Recovery: 7.69 mg. MW by MALDI mass spectral analysis was 196, 803.

### EXAMPLE 2

### Biodistribution of a Pre-mixed Complex Comprising an Indium-Labeled Carboxylesterase-DTPA conjugate and the bsMAb hMN-14×734 (IgG×Fab′)

This example demonstrates the biodistribution of a pre-mixed complex of hMN-14×734 Fab′ (hMN-14 is a humanized antibody of MN-14 (carcinoembryonic antigen; anti-CEA), and an indium-DTPA-carboxylesterase conjugate. Carboxylesterase-DTPA is radiolabeled for tagging purposes with indium-111 radionuclide, using commercially available In-111 acetate. Briefly, In-111 chloride was buffered with 3-times the volume of 0.5 M sodium acetate, pH 6.1; 0.12 mg of CE-DTPA was mixed with 0.25 mCi of In-111 acetate, and incubated for 40 minutes. Then added ˜0.01 mL of cold indium acetate [In acetate: prepared from 0.005 mL of 1.97×10⁻² M indium chloride, 0.045 mL of water and 0.15 mL of 0.5 M sodium acetate, pH 6.1.] After 20 minutes, the solution was made 10 mM in EDTA, incubated for 10 min. ITLC analyses showed 98% of radioactivity associated with carboxylesterase. The pre-mixed hMN-14×734 Fab′/In-111-In-DTPA-carboxylesterase complex is administered to hamsters and nude mice bearing GW-39 human tumor xenografts. Tables 1-6 show that the binding between of the carboxylesterase-DTPA conjugate and the corresponding bispecific antibody is stable in vivo, and that the In-111/In-DTPA-carboxylesterase conjugate can be effectively localized and retained at the tumor sites by its complexation with the hMN-14×734 (IgG×Fab′) bsAb.

### EXAMPLE 3

### ADEPT Therapy Using a Pre-mixed Complex Comprising an Indium (In)-DTPA Carboxylesterase Conjugate and the bsMAb hMN-14×734 (IgG×Fab′)

Male hamsters (body weight: ˜75 g) are given GW-39 human tumor xenografts by injection of a 20% v/v GW-39 tumor cell suspension intramuscularly on the animals' right thigh. After 3 days, a 2:1 premixed complex of mMN-14 F(ab)₂×m734Fab′ and Indium-DTPA-carboxylesterase, at a dose of 0.75 mg of bsAb, corresponding to 200 enzyme units per kg body weight, is administered. Five days post-injection of bsAb/In-DTPA-carboxylesterase, a maximum tolerated dose (8 mg/˜75 g body weight; determined earlier) of the prodrug, CPT-11, is given. A positive control group is given CPT-11 alone and an untreated group are also included in the study. Tumor growth in untreated animals is out of control at 3-4 weeks post-implantation of tumor cells, and animals are sacrificed for humane reasons. Mean tumor volumes are similar for the bsAb/In-DTPA-carboxylesterase and the positive control (CPT-11 alone) at 5 weeks, and out to 9 weeks post-implantation of tumor cells. However, the bsAb/In-DTPA-carboxylesterase treated group continues to show growth inhibition over the next five weeks, while the mean tumor volumes for the group given CPT-11 alone increase during the same period. The relative mean tumor volume for the bsAb/In-DTPA-carboxylesterase treated group at week 14 is similar to the mean tumor volume at week 9 for the positive control, CPT-11 -alone-treated animals. This demonstrates a 5-week advantage in tumor growth control when applying an ADEPT approach using bsAb/In-DTPA-carboxylesterase pretargeting.

### EXAMPLE 4

### Preparation of Carboxylesterase-IMP222 (“CE-IMP222”)

IMP222 is a di-DTPA-containing peptide with the cysteine thiol available for conjugation to maleimide-appended carboxylesterase. IMP222: Ac-Cys-Lys(DTPA)-Tyr-Lys(DTPA)-NH₂. Carboxylesterase (0.0245 umol) was derivatized with a 17.5-fold molar excess of sulfo-SMCC [sulfosuccinimidyl 4(N-maleimidomethyl)-1-cycclohexane carboxylate] in 0.1 M sodium phosphate, pH 7.3, at the ambient temperature for 45 minutes. The product was purified on a 2-mL centrifuged SE column (‘spin column’) of Sephadex G50/80 in 0.1 M sodium phosphate, pH 7.3. The solution of the product was made 1 mM in EDTA, and reacted with a 20-fold molar excess of IMP-222 contained in 0.1 M sodium phosphate-S mM EDTA, pH 6.5, for 45 minutes at the ambient temperature. The product was purified by ‘spin column’ of Sephadex G50/80 in 0.1 M sodium phosphate, pH 7.3. Metal binding analysis using excess of indium acetate spiked with radioactive indium, gave an aveage of 4.5 DTPAs/conjugate in two determinations, or avearge of 2.25 IMP222 moieties per conjugate. Test labeling with In-111 acetate gave 94% incorporation as assayed by ITLC. The material was completely complexed by mixing with a 5-fold molar excess of F6×734 Fab′ Fab′ bispecific antibody, as judged by the shift of the HPLC peak to the higher MW region of the complex.

TABLE 1
Biodistributions of 2:1 pre-mixed complex of [¹²⁵I]-hMN-14
IgG × 734 Fab′ [IgG × Fab′] bispecific antibody
(“¹²⁵I-BsAb”) and [¹³¹I]-In-DTPA-carboxylesterase
(“¹³¹I-CE-DTPA”) in hamsters bearing GW-39 human
tumor xenografts

| | % Injected dose of radioactivity per gram of tissue | | | |
|---|---|---|---|---|
| Tissue | 24 h | 48 h | 120 h | 168 h |
| Tumor: | | | | |
| ¹²⁵I-BsAb | 1.34 ± 0.51 | 2.79 ± 2.21 | 2.60 ± 1.55 | 1.52 ± 0.46 |
| ¹³¹I-CE-DTPA | 0.72 ± 0.40 | 1.23 ± 0.92 | 0.93 ± 0.55 | 0.55 ± 0.26 |
| Liver: | | | | |
| ¹²⁵I-BsAb | 0.86 ± 0.68 | 0.40 ± 0.06 | 0.10 ± 0.03 | 0.11 ± 0.04 |
| ¹³¹I-CE-DTPA | 0.62 ± 0.52 | 0.28 ± 0.04 | 0.07 ± 0.02 | 0.06 ± 0.03 |
| Spleen: | | | | |
| ¹²⁵I-BsAb | 0.66 ± 0.27 | 0.46 ± 0.12 | 0.17 ± 0.06 | 0.13 ± 0.05 |
| ¹³¹I-CE-DTPA | 0.43 ± 0.20 | 0.27 ± 0.07 | 0.13 ± 0.07 | 0.08 ± 0.03 |
| Kidney: | | | | |
| ¹²⁵I-BsAb | 0.72 ± 0.47 | 0.40 ± 0.10 | 0.15 ± 0.02 | 0.16 ± 0.05 |
| ¹³¹I-CE-DTPA | 0.49 ± 0.33 | 0.24 ± 0.04 | 0.07 ± 0.02 | 0.08 ± 0.02 |
| Lungs: | | | | |
| ¹²⁵I-BsAb | 5.57 ± 7.48 | 0.77 ± 0.18 | 0.16 ± 0.05 | 0.18 ± 0.06 |
| ¹³¹I-CE-DTPA | 2.59 ± 3.10 | 0.40 ± 0.12 | 0.06 ± 0.03 | 0.07 ± 0.04 |
| Blood: | | | | |
| ¹²⁵I-BsAb | 2.93 ± 1.67 | 1.75 ± 0.29 | 0.37 ± 0.05 | 0.43 ± 0.11 |
| ¹³¹I-CE-DTPA | 1.86 ± 1.10 | 0.98 ± 0.21 | 0.11 ± 0.08 | 0.19 ± 0.10 |

TABLE 2
Biodistributions of 2:1 pre-mixed complex of [¹²⁵I]-MN-14
F(ab′)₂ × 734 Fab′ [F(ab′)₂ × Fab] bispecific
antibody (“¹²⁵I-BsAb”) and [¹³¹I]-In-DTPA-carboxylesterase
(“¹³¹I-CE-DTPA”) in hamsters bearing GW-39 human tumor xenografts

| | % Injected dose of radioactivity per gram of tissue | | | | |
|---|---|---|---|---|---|
| Tissue | 24 h | 48 h | 72 h | 96 h | 168 h |
| Tumor: | | | | | |
| ¹²⁵I-BsAb | 2.01 ± 1.20 | 2.13 ± 1.28 | 1.01 ± 0.79 | 0.98 ± 0.33 | 0.18 ± 0.05 |
| ¹³¹I-CE-DTPA | 1.07 ± 0.64 | 1.15 ± 0.75 | 0.57 ± 0.45 | 0.56 ± 0.19 | 0.13 ± 0.04 |
| Liver: | | | | | |
| ¹²⁵I-BsAb | 0.18 ± 0.05 | 0.16 ± 0.03 | 0.09 ± 0.05 | 0.05 ± 0.02 | 0.02 ± 0.00 |
| ¹³¹I-CE-DTPA | 0.16 ± 0.04 | 0.15 ± 0.02 | 0.09 ± 0.04 | 0.05 ± 0.02 | 0.02 ± 0.01 |
| Spleen: | | | | | |
| ¹²⁵I-BsAb | 0.25 ± 0.09 | 0.18 ± 0.07 | 0.14 ± 0.12 | 0.09 ± 0.04 | 0.03 ± 0.03 |
| ¹³¹I-CE-DTPA | 0.20 ± 0.05 | 0.13 ± 0.04 | 0.12 ± 0.10 | 0.08 ± 0.03 | 0.03 ± 0.02 |
| Kidney: | | | | | |
| ¹²⁵I-BsAb | 0.03 ± 0.08 | 0.26 ± 0.04 | 0.13 ± 0.06 | 0.06 ± 0.02 | 0.02 ± 0.00 |
| ¹³¹I-CE-DTPA | 0.21 ± 0.08 | 0.17 ± 0.03 | 0.10 ± 0.05 | 0.04 ± 0.02 | 0.02 ± 0.01 |
| Lungs: | | | | | |
| ¹²⁵I-BsAb | 4.47 ± 6.46 | 0.31 ± 0.08 | 0.22 ± 0.17 | 0.07 ± 0.03 | 0.01 ± 0.00 |
| ¹³¹I-CE-DTPA | 5.20 ± 8.93 | 0.21 ± 0.06 | 0.17 ± 0.15 | 0.05 ± 0.04 | 0.01 ± 0.01 |
| Blood: | | | | | |
| ¹²⁵I-BsAb | 0.82 ± 0.36 | 0.84 ± 0.16 | 0.34 ± 0.19 | 0.13 ± 0.06 | 0.02 ± 0.01 |
| ¹³¹I-CE-DTPA | 0.62 ± 0.24 | 0.64 ± 0.11 | 0.30 ± 0.16 | 0.10 ± 0.08 | 0.03 ± 0.02 |

TABLE 3
Biodistributions of 2:1 pre-mixed complex of [¹²⁵I]-hMN-14
Fab′ × 734 Fab′ [Fab′ × Fab′] bispecific
antibody (“¹²⁵I-BsAb”) and [¹³¹I]-In-DTPA-
carboxylesterase (“¹³¹I-CE-DTPA”) in hamsters bearing
GW-39 human tumor xenografts

| | % Injected | | |
|---|---|---|---|
| | dose of radioactivity per gram of tissue | | |
| Tissue | 4 h | 24 h | 48 h |
| Tumor: | | | |
| ¹²⁵I-BsAb | 0.95 ± 0.53 | 1.38 ± 0.33 | 0.15 ± 0.02 |
| ¹³¹I-CE-DTPA | 0.69 ± 0.36 | 0.68 ± 0.14 | 0.13 ± 0.06 |
| Liver: | | | |
| ¹²⁵I-BsAb | 1.15 ± 0.23 | 0.24 ± 0.02 | 0.10 ± 0.00 |
| ¹³¹I-CE-DTPA | 0.75 ± 0.17 | 0.20 ± 0.02 | 0.13 ± 0.01 |
| Spleen: | | | |
| ¹²⁵I-BsAb | 1.14 ± 0.05 | 0.25 ± 0.04 | 0.11 ± 0.02 |
| ¹³¹I-CE-DTPA | 0.75 ± 0.03 | 0.15 ± 0.02 | 0.12 ± 0.04 |
| Kidney: | | | |
| ¹²⁵I-BsAb | 1.20 ± 0.22 | 0.35 ± 0.06 | 0.11 ± 0.02 |
| ¹³¹I-CE-DTPA | 0.87 ± 0.17 | 0.21 ± 0.04 | 0.08 ± 0.02 |
| Lungs: | | | |
| ¹²⁵I-BsAb | 1.38 ± 0.27 | 0.37 ± 0.03 | 0.13 ± 0.02 |
| ¹³¹I-CE-DTPA | 1.00 ± 0.19 | 0.24 ± 0.03 | 0.12 ± 0.02 |
| Blood: | | | |
| ¹²⁵I-BsAb | 5.15 ± 0.96 | 1.27 ± 0.33 | 0.29 ± 0.02 |
| ¹³¹I-CE-DTPA | 3.67 ± 0.69 | 0.94 ± 0.21 | 0.34 ± 0.02 |

TABLE 4
Biodistributions of 2:1 pre-mixed complex of [¹²⁵I]-hMN-14
Fab′ × 734 Fab′ [Fab′ × Fab′] bispecific
antibody (“¹²⁵I-BsAb”) and [¹³¹I]-In-DTPA-
carboxylesterase (“¹³¹I-CE-DTPA”) in nude mice bearing
GW-39 human tumor xenografts

| | % Injected | | |
|---|---|---|---|
| | dose of radioactivity per gram of tissue | | |
| Tissue | 4 h | 24 h | 48 h |
| Tumor: | | | |
| ¹²⁵I-BsAb | 5.87 ± 2.29 | 4.21 ± 0.78 | 2.78 ± 0.56 |
| ¹³¹I-CE-DTPA | 2.53 ± 0.80 | 1.30 ± 0.36 | 1.36 ± 0.41 |
| Liver: | | | |
| ¹²⁵I-BsAb | 3.78 ± 0.84 | 0.20 ± 0.03 | 0.07 ± 0.04 |
| ¹³¹I-CE-DTPA | 2.92 ± 0.62 | 0.29 ± 0.05 | 0.14 ± 0.05 |
| Spleen: | | | |
| ¹²⁵I-BsAb | 8.82 ± 5.82 | 0.34 ± 0.09 | 0.30 ± 0.44 |
| ¹³¹I-CE-DTPA | 7.17 ± 3.34 | 0.64 ± 0.34 | 0.79 ± 0.68 |
| Kidney: | | | |
| ¹²⁵I-BsAb | 8.80 ± 1.09 | 0.40 ± 0.11 | 0.13 ± 0.01 |
| ¹³¹I-CE-DTPA | 3.92 ± 0.77 | 0.26 ± 0.09 | 0.10 ± 0.02 |
| Lungs: | | | |
| ¹²⁵I-BsAb | 5.04 ± 1.27 | 0.33 ± 0.09 | 0.09 ± 0.01 |
| ¹³¹I-CE-DTPA | 4.56 ± 1.45 | 0.29 ± 0.07 | 0.10 ± 0.02 |
| Blood: | | | |
| ¹²⁵I-BsAb | 11.45 ± 1.94 | 0.38 ± 0.11 | 0.07 ± 0.02 |
| ¹³¹I-CE-DTPA | 12.73 ± 3.55 | 0.52 ± 0.15 | 0.17 ± 0.02 |

TABLE 5
Biodistributions of 2:1 pre-mixed complex of [¹²⁵I]-F6
Fab′ (an anti-CEA antibody) × 734 Fab′ bispecific
antibody (“¹²⁵I-BsAb”) [Fab′ × Fab′]
and [¹³¹I]-In-DTPA-carboxylesterase (“¹³¹I-CE-DTPA”)
in nude mice bearing GW-39 human tumor xenografts

| | % Injected dose of radioactivity per gram of tissue | | | |
|---|---|---|---|---|
| Tissue | 4 h | 24 h | 48 h | 72 h |
| Tumor: | | | | |
| ¹²⁵I-BsAb | 4.96 ± 1.19 | 10.01 ± 3.97 | 8.99 ± 2.67 | 11.54 ± 4.06 |
| ¹³¹I-CE-DTPA | 3.28 ± 0.91 | 3.54 ± 1.46 | 3.24 ± 1.02 | 4.50 ± 1.47 |
| Liver: | | | | |
| ¹²⁵I-BsAb | 5.15 ± 0.53 | 1.61 ± 0.22 | 0.78 ± 0.12 | 0.41 ± 0.11 |
| ¹³¹I-CE-DTPA | 4.10 ± 0.42 | 1.16 ± 0.16 | 0.70 ± 0.10 | 0.47 ± 0.09 |
| Spleen: | | | | |
| ¹²⁵I-BsAb | 10.3 ± 1.65 | 2.43 ± 0.60 | 1.15 ± 0.31 | 0.56 ± 0.12 |
| ¹³¹I-CE-DTPA | 6.68 ± 1.15 | 1.37 ± 0.32 | 0.83 ± 0.20 | 0.53 ± 0.11 |
| Kidney: | | | | |
| ¹²⁵I-BsAb | 8.25 ± 0.75 | 2.98 ± 0.31 | 1.06 ± 0.20 | 0.69 ± 0.19 |
| ¹³¹I-CE-DTPA | 5.41 ± 0.32 | 1.64 ± 0.16 | 0.72 ± 0.10 | 0.56 ± 0.14 |
| Lungs: | | | | |
| ¹²⁵I-BsAb | 8.57 ± 2.68 | 3.99 ± 1.81 | 1.65 ± 0.24 | 0.83 ± 0.18 |
| ¹³¹I-CE-DTPA | 6.85 ± 0.44 | 2.21 ± 1.04 | 1.21 ± 0.17 | 0.78 ± 0.20 |
| Blood: | | | | |
| ¹²⁵I-BsAb | 28.54 ± 1.81 | 11.82 ± 1.25 | 5.24 ± 0.78 | 2.48 ± 0.76 |
| ¹³¹I-CE-DTPA | 21.35 ± 1.23 | 7.59 ± 0.76 | 3.88 ± 0.51 | 2.26 ± 0.49 |

TABLE 6
Biodistributions of 2:1 pre-mixed complex of [¹²⁵I]-F6
Fab′ (an anti-CEA antibody) × 734 Fab′ bispecific
antibody (“¹²⁵I-BsAb”) [Fab′ × Fab′]
and [¹³¹I]-In-IMP222-carboxylesterase (“¹³¹I-CE-IMP222”)
in nude mice bearing GW-39 human tumor xenografts

| | % Injected dose of radioactivity per gram of tissue | | | |
|---|---|---|---|---|
| Tissue | 4 h | 25 h | 48 h | 120 h |
| Tumor: | | | | |
| ¹²⁵I-BsAb | 5.17 ± 2.10 | 11.51 ± 1.73 | 11.08 ± 4.37 | 3.66 ± 1.26 |
| ¹³¹I-CE-IMP222 | 2.83 ± 1.06 | 3.96 ± 0.71 | 4.41 ± 1.71 | 2.42 ± 0.75 |
| Liver: | | | | |
| ¹²⁵I-BsAb | 4.84 ± 0.85 | 1.59 ± 0.12 | 0.62 ± 0.08 | 0.16 ± 0.03 |
| ¹³¹I-CE-IMP222 | 3.72 ± 0.63 | 1.14 ± 0.05 | 0.50 ± 0.07 | 0.19 ± 0.03 |
| Spleen: | | | | |
| ¹²⁵I-BsAb | 10.89 ± 3.25 | 2.46 ± 0.92 | 0.89 ± 0.09 | 0.20 ± 0.05 |
| ¹³¹I-CE-IMP222 | 6.98 ± 2.28 | 1.44 ± 0.43 | 0.61 ± 0.08 | 0.22 ± 0.06 |
| Kidney: | | | | |
| ¹²⁵I-BsAb | 7.53 ± 1.90 | 2.38 ± 0.39 | 1.01 ± 0.16 | 0.12 ± 0.02 |
| ¹³¹I-CE-IMP222 | 4.71 ± 1.09 | 1.36 ± 0.23 | 0.68 ± 0.12 | 0.17 ± 0.03 |
| Lungs: | | | | |
| ¹²⁵I-BsAb | 8.55 ± 1.55 | 2.75 ± 0.67 | 0.99 ± 0.16 | 0.07 ± 0.02 |
| ¹³¹I-CE-IMP222 | 6.31 ± 1.18 | 1.80 ± 0.47 | 0.78 ± 0.15 | 0.20 ± 0.03 |
| Blood: | | | | |
| ¹²⁵I-BsAb | 24.41 ± 2.46 | 8.24 ± 0.71 | 2.80 ± 0.26 | 0.17 ± 0.07 |
| ¹³¹I-CE-IMP222 | 21.38 ± 2.87 | 6.93 ± 0.66 | 3.11 ± 0.28 | 0.77 ± 0.19 |

From the foregoing description, one skilled in the art can easily ascertain the essential characteristics of this invention, and without departing from the spirit and scope thereof, can make various changes and modifications of the invention to adapt it to various usage and conditions without undue experimentation. All patents, patent applications and publications cited herein are incorporated by reference in their entirety.

References that may be of Interest:

European Patent No. 0595743.

Auzenne, E., Donato, N. J., Li, C., Leroux, E., Price, R. E., Farquhar, D. and Klostergaard, J. Superior therapeutic profile of poly-L-glutamic acid-paclitaxel copolymer compared with taxol in xenogeneic compartmental models of human ovarian carcinoma. Clin. Cancer Res., 8:573-581, 2002.

Bagshawe, K. D. Antibody-directed enzyme prodrug therapy for cancer: its theoretical basis and application. Mol. Med. Today, 1:424-431, 1995.

Bagshawe, K. D., Sharma, S. K., Springer, C. J., Antoniw, P., Boden, J. A., Rogers, G. T., Burke, P. J., Melton, R. G. and Sherwood, R. F. Antibody directed enzyme prodrug therapy (ADEPT): clinical report. Dis. Markers, 9:233-238, 1991.

Barbet et al., Pretargeting with the affinity enhancement system for radioimmunotherapy. Cancer Biother. Radiopharm., 14:153-166, 1999.

Biela, B. H., Khawli, L. A., Hu, P. and Epstein, A. L. Chimeric TNT-3/human beta-glucuronidase fusion proteins for antibody-directed enzyme prodrug therapy (ADEPT). Cancer Biother, Radiopharm., 18:339-353, 2003.

Bonardi et al., Delivery of saporin to human B-cell lymphoma using bispecific antibody: Targeting via CD22 but not CD19, CD37, or immunoglobulin results in efficient killing. Cancer Res., 53:3015-3021, 1993.

Casey et al., Preparation, characterisation and tumour targeting of cross-linked divalent and trivalent anti-tumour Fab′ fragments. Br. J. Cancer., 74:1397-1405, 1996.

Chen, B. M., Cheng, T. L., Tzou, S. C. and Roffler, S. R. Potentiation of antitumor immunity by antibody-directed enzyme prodrug therapy. Int. J. Cancer, 94:850-858, 2001.

Danks, M. K., Morton, C. L., Krull, E. J., Cheshire, P. J., Richmond, L. B., Naeve, C. W., Pawlik, C. A., Houghton, P. J. and Potter, P. M. Comparison of activation of CPT-11 by rabbit and human carboxylesterases for use in enzyme/prodrug therapy. Clin. Cancer Res., 5:917-924, 1999.

Deckert, P. M., Renner, C., Cohen, L. S., Jungbluth, A., Ritter, G., Bertino, J. R., Old, L. J. and Welt, S. A33scFv-cytosine deaminase: a recombinant protein construct for antibody-directed enzyme-prodrug therapy. Br. J. Cancer, 88:937-939, 2003.

de Bont, D. B., Leenders, R. G., Haisma, H. J., van der Meulen-Muileman, I. and Scheeren, H. W. Synthesis and biological activity of beta-glucuronyl carbamate-based prodrugs of paclitaxel as potential candidates for ADEPT. Bioorg. Med. Chem., 5:405-414, 1997.

de Graaf, M., Boven, E., Scheeren, H. W., Haisma, H. J. and Pinedo, H. M. Beta-glucuronidase-mediated drug release. Curr. Pharm. Des., 8:1391-1403, 2002.

Denny, W. A. Prodrug strategies in cancer therapy. Eur. J. Med. Chem., 36:577-595, 2001.

Francis, R. J., Sharma, S. K., Springer, C., Green, A. J., Hope-Stone, L. D., Sena, L., Martin, J., Adamson, K. L., Robbins, A., Gumbrell, L., O'Malley, D., Tsiompanou, E., Shahbakhti, H., Webley, S., Hochhauser, D., Hilson, A. J., Blakey, D., Begent, R. H. A phase I trial of antibody directed enzyme prodrug therapy (ADEPT) in patients with advanced colorectal carcinoma or other CEA producing tumours. Br. J. Cancer, 87:600-607, 2002.

French. Production of bispecific and trispecific F(ab)₂ and F(ab)₃ antibody derivatives. Methods Mol. Biol., 80:121-134, 1998.

Gestin et al., Two-step targeting of xenografted colon carcinoma using a bispecific antibody and 188Re-labeled bivalent hapten: biodistribution and dosimetry studies. J. Nucl. Med., 42:146-153, 2001.

Hillairet de Boisferon et al., Enhanced targeting specificity to tumor cells by simultaneous recognition of two antigens. Bioconjug Chem., 11:452-460, 2000.

Houba, P. H., Boven, E., van der Meulen-Muileman, I. H., Leenders, R. G., Scheeren, J. W., Pinedo, H. M. and Haisma, H. J. Pronounced antitumor efficacy of doxorubicin when given as the prodrug DOX-GA3 in combination with a monoclonal antibody beta-glucuronidase conjugate. Int. J. Cancer, 91:550-554, 2001.

Jung M. Antibody directed enzyme prodrug therapy (ADEPT) and related approaches for anticancer therapy. Mini Rev. Med. Chem., 1:399-407, 2001.

Kan et al., Thioether-bonded constructs of Fab′gamma and Fc gamma modules utilizing differential reduction of interchain disulfide bonds. J Immunol., 166:1320-1326, 2001.

LeDoussal et al., Enhanced in vivo targeting of an asymmetric bivalent hapten to double-antigen positive mouse B cells with monoclonal antibody conjugate cocktails. J. Immunol., 146:169-175, 1991.

Li, C., Yu, D. F., Newman, R. A., Cabral, F., Stephens, L. C., Hunter, N., et al. Complete regression of well-established tumors using a novel water-soluble poly(L-glutamic acid)-paclitaxel conjugate. Cancer Res., 58:2404-2409, 1998.

Melton, R. G., Boyle, J. M., Rogers, G. T., Burke, P., Bagshawe, K. D. and Sherwood, R. F. Optimisation of small-scale coupling of A5B7 monoclonal antibody to carboxypeptidase G2. J. Immunol Methods, 158:49-56, 1993.

Schott et al., Preparation, characterization, and in vivo biodistribution properties of synthetically cross-linked multivalent antitumor antibody fragments. Bioconjug. Chem., 4:153-165, 1993.

Schoonjans et al., Fab chains as an efficient heterodimerization scaffold for the production of recombinant bispecific and trispecific antibody derivatives. J. Immunol., 165:7050-7057, 2000.

Schoonjans et al., A new model for intermediate molecular weight recombinant bispecific and trispecific antibodies by efficient heterodimerization of single chain variable domains through fusion to a Fab-chain. Biomol. Eng., 17:193-202, 2001.

Sharma, S. K., Bagshawe, K. D., Springer, C. J., Burke, P. J., Rogers, G. T., Boden, J. A., Antoniw, P., Melton, R. G. and Sherwood R F. Antibody directed enzyme prodrug therapy (ADEPT): a three phase system. Dis. Markers, 9:225-231, 1991.

Sharma, S. K., Bagshawe, K. D., Burke, P. J., Boden, R. W. and Rogers, G. T. Inactivation and clearance of an anti-CEA carboxypeptidase G2 conjugate in blood after localisation in a xenograft model. Br. J. Cancer, 61:659-662, 1990.

Spooner, R. A., Friedlos. F., Maycroft, K., Stribbling, S. M., Roussel, J., Brueggen, J., Stolz, B., O'Reilly, T., Wood, J., Matter, A., Marais, R. and Springer, C. J. A novel vascular endothelial growth factor-directed therapy that selectively activates cytotoxic prodrugs. Br. J. Cancer, 88:1622-1630, 2003.

Springer, C. J., Antoniw, P., Bagshawe, K. D. and Wilman, D. E. Comparison of half-lives and cytotoxicity of N-chloroethyl-4-amino and N-mesyloxyethyl-benzoyl compounds, products of prodrugs in antibody-directed enzyme prodrug therapy (ADEPT). Anticancer Drug Des., 6:467-479, 1991.

Springer, C. J. and Niculescu-Duvaz, I. Antibody-directed enzyme prodrug therapy (ADEPT): a review. Adv. Drug Deliv. Rev., 26:151-172, 1997.

Tietze, L. F., Herzig, T., Fecher, A., Haunert, F. and Schuberth, I. Highly selective glycosylated prodrugs of cytostatic CC-1065 analogues for antibody-directed enzyme tumor therapy. Chembiochem. 2:758-765, 2001.

Tietze, L. F., Lieb, M., Herzig, T., Haunert, F. and Schuberth, I. A strategy for tumor-selective chemotherapy by enzymatic liberation of seco-duocarmycin SA-derivatives from nontoxic prodrugs. Bioorg. Med. Chem., 9:1929-1939, 2001.

Werlen et al., Preparation of a trivalent antigen-binding construct using polyoxime chemistry: improved biodistribution and potential for therapeutic application. Cancer Res., 56:809-815, 1996.

Wolfe, L. A., Mullin, R. J., Laethem, R., Blumenkopf, T. A., Cory, M., Miller, J. F., Keith, B. R., Humphreys, J. and Smith, G. K. Antibody-directed enzyme prodrug therapy with the T268G mutant of human carboxypeptidase A1: in vitro and in vivo studies with prodrugs of methotrexate and the thymidylate synthase inhibitors GW1031 and GW1843. Bioconjug. Chem., 10:38-48, 1999.

Xu, G. and McLeod, H. L. Strategies for enzyme/prodrug cancer therapy. Clin. Cancer Res., 7:3314-3324, 2001.

What is claimed is:

1. A method for treating target cells, tissues or pathogens in a subject comprising administering in sequence:
a) a therapeutically effective amount of a non-covalently bound complex to said subject thereby forming a target-tissue-localized complex;
wherein said non-covalently bound complex comprises a multispecific targeting protein comprising at least one target-binding site and one hapten-binding site, and a hapten-enzyme covalent conjugate;
wherein said at least one target-binding site is capable of binding to at least one complementary binding moiety on the target cells, tissues or pathogens or on a molecule produced by or associated with said target cells, tissues or pathogens; and
wherein said hapten-binding site is non-covalently bound to the hapten-enzyme covalent conjugate;
b) optionally, a clearing agent; and
c) a chemotherapeutic drug or prodrug, capable of being converted to a more active drug by the target-tissue-localized complex.

2. The method of claim 1, wherein said multispecific targeting protein is a multispecific antibody or a multispecific antibody fragment.

3. The method of claim 1, wherein said multispecific targeting protein is multivalent.

4. The method of claim 3, wherein said multivalent, multispecific targeting protein is an anti-CEA×anti-indium-DTPA F(ab′)₂×Fab′.

5. The method of claim 1, wherein said multispecific targeting protein is at least bispecific.

6. The method of claim 1, wherein said complex is injected intravenously, intravesically, intra-arterially, intra-tumorally or intraperitoneally into said subject.

7. The method of claim 1, wherein said complex binds to a cellular tumor-associated antigen.

8. The method of claim 7, wherein the cellular tumor-associated antigen is selected from AFP, EGP-1, EGP-2, CD37, CD74, colon-specific antigen-p (CSAp), carcinoembryonic antigen (CEA), CD19, CD20, CD21, CD22, CD23, CD30, CD37, CD74, CD80, HLA-DR, HCG, Ia, MUC 1, MUC 2, MUC 3, MUC 4, EGFR, HER 2/neu, PAM-4, TAG-72, EGP-1, EGP-2, A3, KS-1, Le(y), S100, PSMA, PSA, tenascin, folate receptor, VEGFR, necrosis antigens, IL-2, T101 and MAGE9.

9. The method of claim 1, wherein said hapten-enzyme conjugate comprises at least one hapten.

10. The method of claim 9, wherein said hapten is selected from the group consisting of HSG, DTPA, indium-DTPA, DOTA, indium-DOTA, yttrium-DOTA, fluorescein or biotin.

11. The method of claim 9, wherein two haptens are linked by a peptide of from 2-10 amino acid residues in length.

12. The method of claim 9, wherein two haptens are linked by a peptide of from 2-5 amino acid residues in length.

13. The method of claim 9, wherein two haptens are linked by a peptide of 3 amino acid residues in length.

14. The method of claim 9, wherein the haptens are attached via a single reaction site to the enzyme.

15. The method of claim 1, wherein the enzyme is an esterase, amidase, glucuronidase or a galactosidase.

16. The method of claim 15, wherein the enzyme is a carboxylesterase.

17. The method of claim 16, wherein the carboxylesterase is rat, mouse, rabbit, porcine or human carboxylesterase.

18. The method of claim 15, wherein the enzyme is produced by recombinant techniques.

19. The method of claim 18, wherein the enzyme is produced in yeast, bacteria, plants, insect cells or animals.

20. The method of claim 15, wherein the enzyme has been modified to enhance its catalytic properties.

21. The method of claim 20, wherein the enzyme is modified by site-directed mutagenesis.

22. The method of claim 20, wherein the modification increases the rate of enzyme-substrate catalysis and/or reduces the Michaelis constant of the enzyme.

23. The method of claim 1, wherein the multispecific targeting protein binds to both its antigenic target and to its hapten target with an dissociation constant of at least 10⁻⁷, more preferably at least 10⁻⁹.

24. The method of claim 1, wherein the multispecific targeting protein is murine, chimeric, humanized, human, or a mixture of proteinaceous components from this list.

25. The method of claim 1, wherein the optional clearing agent is an antibody directed against an epitope of the multispecific targeting protein/hapten-enzyme complex.

26. The method of claim 1, wherein the optional clearing agent is an anti-idiotypic antibody to the multispecific targeting protein

27. The method of claim 24, further comprising a carbohydrate-derivatized anti-idiotypic antibody to the multispecific targeting protein.

28. The method of claim 25, further comprising a galactosylated anti-idiotypic antibody to the multispecific targeting protein.

29. The method of claim 1, wherein the chemotherapeutic prodrug has greater aqueous solubility than the active drug produced by the multispecific targeting protein.

30. The method of claim 1, wherein the chemotherapeutic prodrug is a prodrug of a camptothecin, doxorubicin, taxol, actinomycin, maytansine, calicheamicin or epithilone class of drug.

31. The method of claim 30, wherein the chemotherapeutic prodrug is a prodrug of SN-38.

32. The method of claim 30, wherein the chemotherapeutic prodrug is CPT-11.

33. The method of claim 1, wherein said pathogen is a virus, a fungus, a parasite or bacteria.

34. The method of claim 1, where said subject is a mammal.

35. The method of claim 34, wherein said mammal is a human.

36. A kit comprising, in suitable containers:
a) a multispecific targeting protein, comprising at least one target-binding site and a hapten-binding site, pre-mixed with a hapten-enzyme conjugate; and
b) a chemotherapeutic prodrug,
wherein a) and/or b) optionally further comprise a pharmaceutically acceptable carrier.

37. A kit comprising, in separate, suitable containers:
a) a multispecific targeting protein, comprising at least one target-binding site and a hapten-binding site;
b) a hapten-enzyme conjugate; and
c) a chemotherapeutic prodrug;
wherein said multispecific targeting protein, comprising at least one target-binding site and a hapten-binding site and said hapten-enzyme conjugate are mixed immediately prior to use,
wherein a), b) and/or c) optionally further comprise a pharmaceutically acceptable carrier.

38. A method of making a stable non-covalently bound complex that is capable of localizing to a target cell, tissue, or pathogen comprising admixing a multispecific targeting protein comprising at least one target-binding site and a hapten-binding site, and a hapten-enzyme covalent conjugate;
wherein said at least one target-binding site is capable of binding to at least one complementary binding moiety on said target cells, tissues or pathogens or on a molecule produced by or associated with said target cells, tissues or pathogens; and
wherein said hapten-binding site is capable of stably and non-covalently binding said hapten-enzyme conjugate; thereby making a stable non-covalently bound complex.

39. A method of treating a subject, comprising administering a therapeutically effective amount of a non-covalently bound complex, said non-covalently bound complex resulting from the pre-mixing of said multi-specific targeting protein and a hapten-enzyme conjugate, prior to administration to said subject.

40. The method of claim 1, where said subject is a mammal.

41. The method of claim 40, wherein said mammal is a human.
